(19) United States
(12) Patent Application Publication
(10) Pub. No.: US 2022/0319680 A1
(43) Pub. Date: Oct. 6, 2022

(54) SYSTEMS AND METHODS FOR FACILITATING HEALTHCARE

(71) Applicant: TOTIUM PTY LTD, Sydney (AU)

(72) Inventor: Symone RYAN, Sydney (AU)

(21) Appl. No.: 17/620,597

(22) PCT Filed: Jun. 17, 2020
(86) PCT No.: PCT/AU2020/050609

§ 371 Date: Dec. 17, 2021

(30) Foreign Application Priority Data
Jun. 18, 2019 (AU) 2019902120

(51) Int. Cl. G16H 40/20; G16H 80/00; G06Q 10/10; G06Q 30/06

(52) U.S. Cl. CPC G16H 40/20; G16H 80/00; G06Q 10/1095; G06Q 30/0641

(57) ABSTRACT

Described herein is a computer implemented method including: receding. by a health management system, patient health data in respect of a patient; analysing, by the health management system, the patient health data to determine that engagement with a particular type of healthcare professional is required; determining, by the health management system, a specific healthcare professional of the particular type, causing, by the health management system, a healthcare professional engagement interface to be generated, the healthcare professional engagement interface including information indicating that engagement with the particular type of healthcare professional is required and a do not contact control, and in response to determining that the do not contact control is not activated, causing a patient contact reminder to be generated for the specific healthcare professional, the patient contact reminder being a reminder for the specific healthcare professional to call the patient.

### FIELD OF THE DISCLOSURE

The present disclosure generally relates to systems and methods for facilitating healthcare, and in particular to systems and methods for reducing the action required by a patient to further his or her healthcare.

### BACKGROUND

Healthcare is an issue of fundamental and critical importance. Improving access to healthcare provides benefits to both individuals and society as a whole.

n the majority of situations—particularly with respect to preventative healthcare—individual initiative and action is required in order to access healthcare services. The initiative and action required differ depending on the care in question. For example, an individual may need to arrange and attend a pathology clinic, doctor or other healthcare professional, fill a pharmaceutical script, engage in/commit to a health program (e.g. exercise program), or simply improve their knowledge of things to do/not to do to improve or maintain their health.

Given the obvious benefits of staying healthy, taking the initiative and performing actions to improve (or at least maintain) health should be a fundamental priority for a given person. Unfortunately, however, this is often not the case. In many cases, people lead busy lives with numerous time pressures, and taking care of oneself does not assume the priority it should. For many individuals even something as simple as organizing and attending a doctor's appointment is the type of thing that is put off until it becomes essential—i.e. the individual is unable to function at what they consider an acceptable level. At this point, however, what may have been a simple issue to deal with had it been addressed early on may have evolved into a far more significant issue, requiring greater time and more resources to correct (if, in fact, correction is still possible at the later stage).

Given this, there is significant value in reducing the friction associated both with educating individuals (which can lead to healthcare taking a higher priority) and in providing healthcare services to individuals.

Various technology tools are available that assist—to a certain extent—in simplifying access to healthcare. For example, some medical centres/doctors surgeries provide either web-based or app-based booking system in an attempt to make booking an appointment simpler. Similarly, obtaining healthcare products has, arguably, become simpler with the rise of ecommerce systems and online ordering. These solutions, however, still rely on the individual taking the initiative to make an appointment.

### SUMMARY

In one aspect, the present invention provides a computer implemented method including: receiving, by a health management system, patient health data in respect of a patient; analysing, by the health management system, the patient health data to determine that engagement with a particular type of healthcare professional is required; determining, by the health management system, a specific healthcare professional of the particular type; causing, by the health management system, a healthcare professional engagement interface to be generated, the healthcare professional engagement interface including information indicating that engagement with the particular type of healthcare professional is required and a do not contact control; in response to determining that the do not contact control is not activated, causing a patient contact reminder to be generated for the specific healthcare professional, the patient contact reminder being a reminder for the specific healthcare professional to call the patient.

In a second aspect, the present invention provides a computer implemented method including: receiving, by a health management system, patient health data in respect of a patient; analysing, by the health management system, the patient health data to determine that a healthcare product is required; determining, by the health management system, a specific provider for the healthcare product; causing, by the health management system, a healthcare product order interface to be generated, the healthcare product order interface including information regarding the healthcare product and a do not order control; in response to determining that the do not order control is not activated, generating a product order and communicating the product order to a provider system of the specific provider.

### BRIEF DESCRIPTION OF THE DRAWINGS

In the drawings:

FIG. 1 is a diagram depicting networked computer processing systems and an environment in which the embodiments described herein can be implemented.

FIG. 2 is a block diagram of a computer processing system configurable to perform embodiments described herein.

FIG. 3 is a flowchart depicting operations performed to acquire and analyse patient health data in accordance with an embodiment.

FIG. 4 is a flowchart depicting operations performed to generate health report interface data in accordance with an embodiment.

FIG. 5 is an example health report interface in accordance with an embodiment.

### DETAILED DESCRIPTION

The embodiments described herein operate to simplify access to healthcare services. Generally speaking, this is achieved by configuring computer systems to operate (and in some cases interoperate) so as to reduce the action required by a patient to further his or her healthcare and/or increase their motivation/commitment to doing so.

Initially, a high level overview of the various systems involved which are configured to operate in the embodiments described herein will be provided. This is followed by a description of a computer processing system which may be configured to perform various operations and functions as described herein. Following this, the operations performed by the various systems will be described.

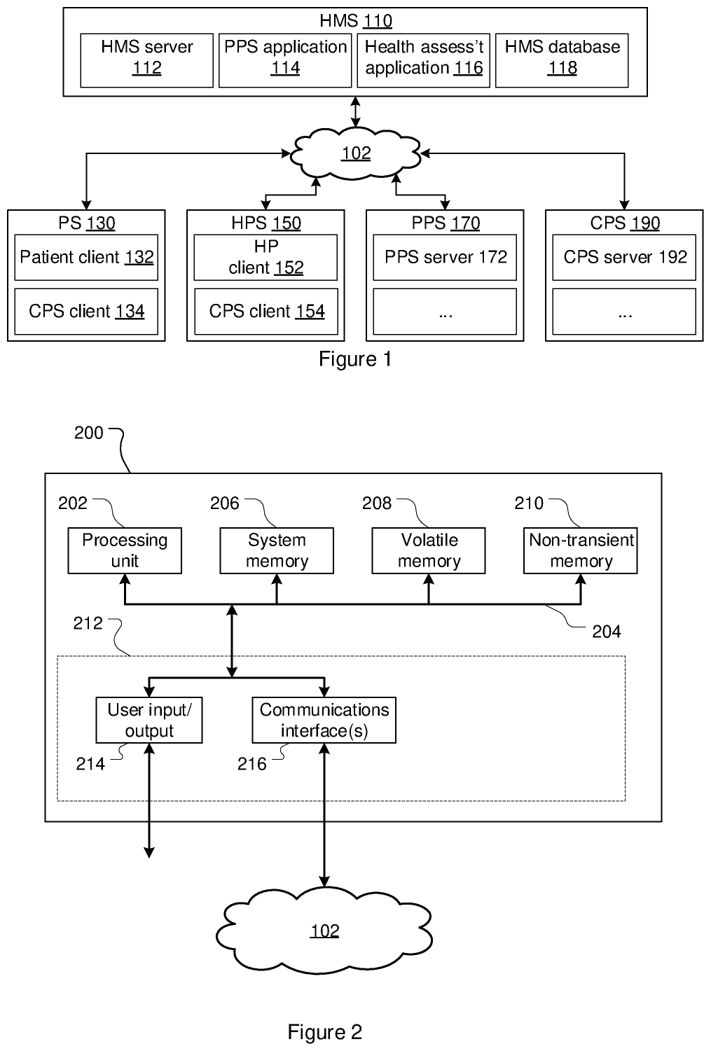

Turning to FIG. 1, a networked environment 100 including various computer processing systems involved in various aspects of the present disclosure will be described.

Environment 100 includes a healthcare management system 110, a patient system 130, a healthcare professional system 150, a product provider system 170, and a content provider system 190. These systems are interconnected via one or more telecommunications networks 102.

For brevity, the following acronyms are at times used in this specification: HMS for healthcare management system; PS for patient system; HP for healthcare professional; HPS for healthcare professional system; PPS for product provider system; CP for content provider; and CPS for content provider system.

The HMS 110 is a computer processing system (or group of computer processing systems) that, functionally speaking, includes a HMS server 112, a PPS application 114, a health assessment application 116, and a HMS database 118.

HMS server application 112 (which will also be referred to as the HMS sever 112 for ease of reference) is a software application that, when executed by the HMS 110, configures the HMS 110 to provide server-side functionality for HMS client applications (e.g. patient client 132 running on patient system 130, HP client 152 running on HPS 150).

HMS server 112 can be implemented in various ways. For example, HMS server 112 can be a web server application which executes on the HMS 110 to expose a web-interface to clients (e.g. client 132 or 152). In this case, the relevant client 132/152 is a web browser application or application with web browser functionality. Alternatively, HMS servers 112 can be an application server that executes on the HMS 110 to provide a programmatic interface (API) for clients (e.g. client 132 or 152). In this case, the relevant client 132/152 will be a dedicated healthcare management system client application (e.g. an app), the client and server communicating with one another via defined API calls.

PPS application 114 is a software application that, when executed by the HMS 110, configures the HMS 110 to communicate with a product provider system such as PPS 170. As described further below, the HMS 110 communicates with a PSP in order to place orders for healthcare products (e.g. pharmaceuticals or other healthcare products) on behalf of patients. PPS application 114 can be configured to communicate with a single PPS or multiple different PPSs (or, alternatively, multiple PPS applications 116 may be provided, each providing access to a different PPS).

Health assessment application 116 is a software application that, when executed by the HMS 110, configures the HMS 110 to analyse a patient's health data and generate various health assessment outputs based thereon. In the present disclosure, the health assessment outputs generated by the health assessment application 116 include reporting outputs and action outputs.

Reporting outputs are outputs that are reported to a patient (and/or, in certain cases, a healthcare professional). By way of example, and as discussed further below, reporting outputs can include patient health reports, patient health plans, and patient education plans.

Action outputs are outputs for which the HMS 110 performs additional operations in order to reduce the burden on patients in furthering their healthcare. By way of example, action outputs can include engagement action outputs (which are processed further to facilitate a call or appointment with a healthcare professional) and product order action outputs.

HMS database 118 stores data required by the HMS 110. This may include, for example: user account details such as user names and authentication details (e.g. in respect of patient accounts, healthcare professional accounts, and system administrator accounts): patient data, as obtained, for example, through patient questionnaires and/or from healthcare professionals; healthcare professional data such as contact details and services provided by healthcare professional who interact with the HMS 110; product provider system data required, for example, to connect with and interact with various product provider systems such as PPS 170; health assessment data as used by the health assessment application 118 to analyse patient data and generate reporting and/or action outputs.

While HMS database 118 is depicted as a single database it may, in fact, be several separate databases. For example, sensitive patient data may be stored by a separate database to that (or those) which store other data used by the HMS 110 (e.g. user account/credential data, health assessment data etc.).

In FIG. 1 HMS 110 is depicted as a single system. The functions of the HMS 102 can be performed by multiple computer processing systems communicating data between one another as required.

By way of example, HMS server 112 may run on a separate hardware system to the PPS application 114, health assessment application 116, and/or the HMS database 118. Alternatively, HMS server 112 may, in fact, be multiple server applications (e.g. one dedicated to dealing with patient clients 132 and one dedicated to deadline with HP clients 152).

In addition to patients and healthcare professionals, HMS server 112 provides access to and particular functionality for additional types of users. For example, access for at least administrator users will be provided, through which administrators can configure, update, and perform other administrative actions with respect to the HMS 110.

HMS server 112, PPS application 114, health assessment application 116, and/or HMS database 118 can be configured as scalable systems that are programmed to automatically provision/de-provision resources based on user/system demand.

Patient system 130 is a computer processing system used by a patient to interact with the HMS 110. To facilitate this interaction, the PS 130 includes a patient client 132. Patient client 132 is a software application that, when executed by the PS 130, configures the PS 130 to provide HMS client-side patient functionality.

Healthcare professional system 150 is a computer processing system used by a healthcare professional to interact with the HMS 110. To facilitate this interaction, the HPS 150 includes a HP client 152. HP client 152 is a software application that, when executed by the HPS 150, configures the HPS 150 to provide HMS client-side healthcare professional functionality.

Patient functionality (as provided by the patient client 132) and HP functionality (as provided by the HP client 152) are described further below. Generally speaking, however, client side functionality involves receiving data from and communicating data to the HMS server 112 over communications network 102; generating and displaying (or otherwise providing) user interfaces on the PS 130 or HPS 150; receiving user inputs entered at the PS 130 or HPS 150. Where HMS server 112 is a web server, the corresponding client 132152 will typically be a web browser application, for example Chrome, Safari, Internet Explorer, or other application that communicates with the web server via world-wide-web protocols (e.g. http, https). Alternatively, where HMS server 112 is an application server, the corresponding client 132/152 will be a dedicated application that communicates with the HMS server 112 using defined API communications.

Each of the patient system 130 and HP system 150 will typically be a personal computing device, for example a desktop computer, laptop computer, tablet computer, smart phone, or other computing device. Accordingly, the patient and HP systems 130 and 150 will be provided with/run additional software applications to those illustrated and described. For example, and at the very least, each of the patient and HP systems 130 and 150 will be provided with an operating system to facilitate basic functionality.

Product provider system 170 is a computer processing system operated by an entity that provides for the online ordering of healthcare products. This generally involves receiving orders for healthcare products (and associated information such as payment details and delivery details) and arranging for the fulfilment of those orders. Examples of product provider systems include Amazon, pharmacy ecommerce systems, and other ecommerce systems. PPS 170 includes a PPS server 172 which is configured to receive orders and associated information communicated by the PPS application 114 of the HMS 110. Product provider system 170 will typically (though need not) be owned and operated by a separate entity to the entity that owns/operates the HMS 110, and in order to provide its services will normally include additional functional components to the single PPS server 172 depicted.

Content provider system 190 is a computing system that stores and provides content to users. As described in further detail below, in certain cases a reporting output of the health assessment application 118 for a given patient includes an education plan. In some instances, education plans include reference(s) to content hosted by a CPS 190. To allow access to this content, the CPS 190 includes a CPS sever 192 which receives content requests (from a CPS client such as client 134 operating on a PS 130 or client 142 operating on a HPS 150) and responds thereto. CPS server 192 may be a web server or application server. By way of example, CPS 190 may be any website or application that provides access to healthcare information in any form (e.g. text, images, video, audio).

Although environment 100 has been illustrated with a single patient system 130, single HPS 150, single PPS 170, and single CPS 190, environment 100 will typically include multiple patient systems130 owned/operated by multiple patients, multiple HPSs 150 owned/operated by multiple HPs. Environment 100 may also include multiple product provider systems 170 and/or multiple content provider systems 190.

In environment 100 described above, each of the HMS 110, PS 130, HPS 150, PPS 170, and CPS 190 is a computer processing system (or multiple computer processing systems configured to interoperate to perform the operations and provide the functionality described herein).

FIG. 2 provides a block diagram of a general purpose computer processing system 200 which can be configured to become a HMS 110, PS 130, HPS 150, PPS 170, and/or CPS 190 as described herein.

System 200 includes at least one processing unit 202. The processing unit 202 may be a single computer processing device (e.g. a central processing unit, graphics processing unit, or other computational device), or may include a plurality of computer processing devices. In some instances all processing described as being performed by a given computer processing system 200 is performed by its processing unit 202, however in other instances processing may also be performed by remote processing devices accessible and useable (either in a shared or dedicated manner) by the system 200.

Through a communications bus 204 the processing unit 202 is in data communication with a one or more machine readable storage (memory) devices which store instructions and/or data for controlling operation of the processing system 200. In this instance system 200 includes a system memory 206 (e.g. a BIOS), volatile memory 208 (e.g. random access memory such as one or more DRAM modules), and non-volatile memory 210 (e.g. one or more hard disk drives, solid state drives, or other persistent storage devices).

System 200 also includes one or more interfaces, indicated generally by 212, via which system 200 interfaces with various devices and/or networks. Generally speaking, other devices may be integral with system 200, or may be separate. Where a device is separate from system 200, connection between the device and system 200 may be via wired or wireless hardware and communication protocols, and may be a direct or an indirect (e.g. networked) connection.

Wired connection with other devices/networks may be by any appropriate standard or proprietary hardware and connectivity protocols—e.g. USB; eSATA; Ethernet; HDMI; to name a few (with other protocols being possible).

Wireless connection with other devices/networks may similarly be by any appropriate standard or proprietary hardware and communications protocols—e.g. BlueTooth, WiFi, NFC, GSM, EDGE, LTE, CDMA to name a few (with other protocols being possible).

Generally speaking, the devices to which system 200 connects—whether by wired or wireless means—include one or more input devices to allow data to be input into/received by system 200 for processing by the processing unit 202, and one or more output devices to allow data to be output by system 200. Example devices are described below, however it will be appreciated that not all computer processing systems will include all mentioned devices, and that additional and alternative devices to those mentioned may be used.

For example, system 200 may include or connect to one or more input devices by which information/data is input into (received by) system 200. Such input devices may include keyboards, mice, trackpads, microphones, accelerometers, proximity sensors, GPS devices and the like. System 200 may also include or connect to one or more output devices controlled by system 200 to output information. Such output devices may include display devices (e.g. CRT/LCD/LED/plasma displays), touch screen displays (providing both input and output), speakers, vibration modules, lights, and such like. System 200 may also include or connect to devices which may act as both input and output devices, for example memory devices (hard drives, solid state drives, disk drives, compact flash cards, SD cards and the like) which system 200 can read data from and/or write data to, and touch screen displays which can both display (output) data and receive touch signals (input).

System 200 may also connect to one or more communications networks (e.g. the Internet, a local area network, a wide area network, a personal hotspot etc.) to communicate data to and receive data from networked devices.

System 200 may be any suitable computer processing system such as, by way of non-limiting example, a server computer system, a desktop computer, a laptop computer, a netbook computer, a tablet computing device, a mobile/smart phone, a personal digital assistant, or any other system.

System 200 stores or has access to computer applications (e.g. instructions and data) which, when executed by the processing unit 202, configure system 200 to receive, process, and output data. Such programs typically include an operating system such as Microsoft Windows®, Apple OSX, Apple 105, Android, Unix, or Linux.

System 200 also stores or has access to instructions and data (i.e. applications/software) which, when executed by the processing unit 202, configure system 200 to perform various computer-implemented processes/methods as described herein. Examples of such applications include patient server 112, HPS server 112, PPS application 116, health assessment application 118, HMS database 118, patient client 132, HP client 152, CPS client 134/154, PPS server 172 and CPS server 192 as described above.

Instructions and data are stored on a non-transient machine readable medium accessible to system 200. For example, instructions and data may be stored on non-transient memory 210. Instructions may be transmitted to/received by system 200 via a data signal in a transmission channel enabled (for example) by a wired or wireless network connection.

It will be appreciated that FIG. 2 does not illustrate all functional or physical components of a computer processing system. For example, no power supply or power supply interface has been depicted, however system 200 will either carry a power supply or be configured for connection to a power supply (or both). It will also be appreciated that the particular type of computer processing system will determine the appropriate hardware and architecture, and alternative computer processing systems suitable for implementing aspects of the invention may have additional, alternative, or fewer components than those depicted.

In order to provide the functionality described herein various HMS 110 setup and administrative operations are performed. An overview of these operations will be described in this section, recognizing that many of these operations (e.g. user account creation and management) will be known to a skilled addressee and can be implemented in various ways.

For patients to interact with the HMS 110 patient accounts are created. A patient account includes various information allowing the patient to access and make use of the services provided by the HMS 110. This information includes, for example, a patient username, patient authorization/authentication details (e.g. a password and/or other authentication data), a patient name, patient contact details (e.g. email address, telephone number(s), address(es) etc.). At the time of creating an account, a patient may also provide certain patient demographic data—for example a date of birth and gender.

Patient accounts may be created by patients themselves (accessing the HMS 110 via a patient client 132), by HMS administrators, or a combination thereof (e.g. a HMS administrator creating a patient account and providing logon details to the patient who then completes the account process by providing further patient information).

Either at the time of creating a patient account or over the course of using the HMS 110 patients may provide/update additional patient information and/or define various patient user parameters which impact the operations of the HMS 110 for that patient. For example, the patient may define one or more of: preferred patient contact details; preferred delivery address (for product order purposes); preferred healthcare professional providers for different types of services (e.g. preferred general practitioner, preferred dentist, preferred optometrist, preferred physiotherapist, and/or other preferred specialist); preferred healthcare professional location (e.g. near the patient's home address, work address, or other address); healthcare product preferences (e.g. whether generic products are acceptable substitutes for brand name products or not); payment details (such as credit card or other payment account details); healthcare insurer details; government health program details, and any other relevant preferences/information.

For healthcare professionals to interact with the HMS 110 HP accounts are created. A HP account includes various information allowing the HP to access and make use of the services provided by the HMS 110. This information includes, for example, a HP username, HP authorization/authentication details (e.g. a password and/or other authentication data), a HP name, HP contact details (e.g. email address, telephone number(s), HP address(es) etc.); the type of services offered (e.g. general practitioner services, optometry services, dental services, physiotherapy services, other services); whether the healthcare professional does home/offsite visits and, of so, the area serviced; any other relevant details.

HP accounts may be created by HPs themselves (accessing the HMS 110 via a HP client 152), by HMS administrators, or a combination thereof (e.g. a HMS administrator creating a HP account and providing logon details to the HP who then completes the account process by providing further HP information).

Either at the time of creating a HP account or over the course of using the HMS 110 HPs may provide/update additional HP information and/or define various patient HP user parameters which impact the operations of the HMS 110 for that HP. For example, HPs may provide calendar access data allowing the HMS 110 to access the HPs calendar (e.g. to queue patient calls and/or make appointments).

User account details (including, patient account details, HP account details, HMS administrator account details) are stored for access/use by the HMS 110 as required, for example in HMS database 118.

In certain embodiments the HMS 110 is configured to interact with one or more product provider systems, such as PPS 170. Such configuration may be performed in various ways, for example by storing PPS access credentials (e.g. in the HMS database 118) which are used by the PPS application 116 to interact with the PPS server 172.

HMS 110 also stores query generation data, patient health data, health assessment data, and health education data—e.g. in HMS database 118.

The query generation data is used by the HMS 110 to generate queries which, in turn, are used to acquire patient query response data (one type of patient health data). Generally speaking, acquiring patient query response data involves the HMS server 112 providing patient queries to the patient client 132 which, in turn, causes the queries to be displayed or otherwise presented by the patient system 130. The patient responds to the queries (e.g. using an input device such as a touch screen or keyboard) and the patient client 132 communicates the responses back to the patient server 112 to be saved as patient query response data in the HMS database 118.

The query generation data and queries generated therefrom are typically aimed at gathering various types of information relevant to assessing a patient's health: for example, demographic information, behavioural information, health history information, and pathology/biometric information. Patient demographic information may include, for example, information such as: age; gender; race, income. Patient behaviour information may include, for example, information such as: eating/dietary habits; smoking habits; alcohol consumption habits; drug consumption habits; exercise habits; sleep habits; work habits; stress levels. Health history information may include, for example, information such as: current/prior medical conditions; the patient's family medical history; current medications being taken by the patient.

The query generation data is periodically updated (e.g. by HMS administrator users) as medical research points to other items or types of patient data that are (or may be) relevant to assessing a patient's health.

As noted, patient query response data is one type of patient health data. Other types of patient health data received and stored by the HMS 110 include patient pathology/biometric data (e.g. blood type, blood pressure, blood/sugar measurements, height, weight, waist measurement). Patient pathology data can be submitted to the HMS 110 by patients themselves (via the operation of patient server 112 and patient client 132) and/or by healthcare professionals on behalf of their patients (e.g. doctors, specialists, pathology clinicians, nurses, etc. via the operation of HPS server 112 and HP client 152).

The health assessment data is used to analyse patient health data available to the HMS 110 (e.g. stored on HMS database 118) and generate various outputs based thereon.

As with the query generation data, the health assessment data is periodically updated (e.g. by HMS administrator users) as medical research evolves.

The health education data provides either educational content or links to educational content maintained on other content provider systems (such as CPA 190). Educational content may be in the form of articles, books, webpages, videos, audio, infographics, and/or any other means for conveying information. The health education content is also updated as different education content becomes available.

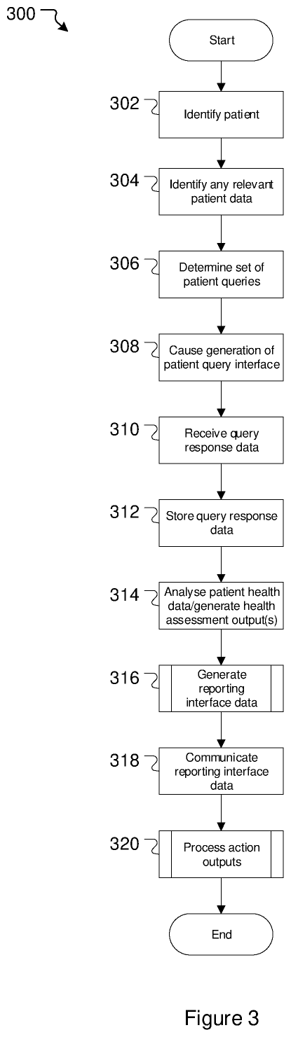

Turning to FIG. 3, operations performed by the HMS 110 in accordance with an embodiment will be described.

The operations of process 300 are performed in the context of a patient user accessing the HMS server 112 (specifically via the patient client 132) to provide patient data and obtain a health assessment report. In certain cases, however, a patient's healthcare professional may access the HMS server 112 (via the HP client 152) to provide patient health data on the patient's behalf.

At 302, HMS server 112 identifies the patient in question. The patient is identified by account details associated with the account used by the patient to logon/access the HMS server 112.

At 304, the HMS server 112 uses retrieves any relevant patient data associated with the identified patient and already stored by the HMS 110 (e.g. in HMS database 118). In some cases, no relevant patient data may be available—for example where the patient is accessing the HMS 110 for the first time and has not yet provided any relevant data. In other cases, a small amount of patient data will already exist—e.g. patient demographic data provided on creation of the patient's account. In still further cases, substantial patient data may already be stored by the HMS 110—for example where the patient has previously been through process 300.

At 306, the HMS server 112 generates a set of queries to be provided to the patient in order to obtain desired patient health data. The set of queries is generated based on the query generation data and any relevant patient health data retrieved at 304. In some cases, e.g. where no patient health data is available, the HSM server 112 generates a default set of queries. In other cases, the HMS server 112 generates a patient specific (or patient type specific) set of queries based on the relevant patient data retrieved at 304. For example, certain queries may or may not be provided to a particular patient depending on their gender and/or age, what queries the patient has already responded to, etc.

As noted above, and generally speaking, the types of queries that can be generated include queries designed to obtain information in respect of the patient's demographic, behaviour, health history, and/or laboratory/biometric information.

Examples of demographic related queries include queries such as: What is your age?; What is your gender?; What is your race?; What is your income level?

Examples of behaviour related queries include: Do you smoke?; Do you drink?; How often do you exercise?

Examples of health history related queries include: Is there a history of disease x in your family?; Have you ever suffered a heart attack?; Are you currently taking any medication?

Examples of laboratory/biometric related queries include: What is your height?; How much do you weigh?; What is your waist measurement?

At 308, the HMS server 112 causes the patient client application 132 to generate a patient query interface on the patient system 130. The patient query interface presents (e.g. displays) the queries determined at 306 and receives responses thereto. Any appropriate query interface may be used, for example a questionnaire type interface which displays queries and associated input controls which can be utilized by the patient to enter responses.

At 310, the HMS server 112 receives query response data from the client application 132—i.e. data generated by the client application 132 in response to the patient responding to the queries presented in the query interface.

At 312, the HMS server 112 stores the query response data in the HMS database 118 (the response data being associated with the patient in question).

At 314, the health assessment application 116 analyses the patient heath data available (e.g. the query response data stored at 312 along with any previously received patient data) and generates one or more outputs. The specific manner in which the HMS 110 analyses the patient health data is not the focus of the present disclosure, and various techniques known in the art may be used in the analysis. For example, the health assessment application 116 may be (or include or make use of) an expert or rule based system that generates outputs based on the patient health data and the health assessment data stored in the HMS database 118.

As noted above, in the present disclosure outputs generated by analysing a patient's health data can include (depending on the patient health data in question and analysis performed) reporting outputs and action outputs. With respect to reporting outputs, and as noted above, the present disclosure explicitly contemplates the generation of patient health reports, patient health plans, and patient education plans. With respect to action outputs, and as also noted above, the present disclosure explicitly contemplates the generation of engagement action outputs, and product order action outputs. The health assessment application 116 can be configured to generate additional and/or alternative reporting outputs and/or action outputs. The specific outputs for a particular patient depend on the patient's health data and the results of the analysis thereof.

A patient health report includes information in respect of a patient's health. This information may include, for example, one or more of:
An overall health indicator such as a score, a colour code (e.g. red for serious and immediate risk, orange for moderate risk, green for low risk) or other indicator.
Specific health area indicators (e.g. scores, colour codes or other indicators) in respect of specific health areas relevant to the patient—for example areas such as: men's health; heart health; mind and mood.
Information on the patient's susceptibility to certain diseases or conditions (e.g. a low/moderate/high susceptibility to disease/condition x).
Patient action items—i.e. actions that the patient can undertake and that will have a beneficial impact on their health. (Such patient actions should not be confused with the ‘action outputs’ described above, the distinction being that the ‘action outputs’ involve further HMS 110 operations (described below), while patient actions are simply information presented to a patient for them to action or not.

In certain embodiments, patient action items are associated with an importance ranking. The importance ranking can be based on various factors, for example the urgency with which an action should ideally be undertaken, the impact of the action, and/or a difficulty (or perceived difficulty) associated with the action. For example, if analysis suggests a certain action x for the patient, but the benefit of the patient performing x is low, this would typically be provided with a low importance ranking (particularly if x has a high difficulty). Conversely, if analysis suggests a certain action y for the patient, and the consequences of the patient failing to performing y is high, this would typically be provided with a high importance ranking. Furthermore, in order to not overwhelm the patient/increase the likelihood of the patient undertaking none of the determined patient action items, a maximum number (e.g. 1, 2, 3, n) of patient action items may be generated (or, downstream, delivered to the patient).

To provide an example, the top three patient action items for a particular patient could be: 1) stop smoking; 2) exercise more; 3) eat less red meat.

A patient health plan includes information on various actions the patient could consider undertaking to improve (or, depending on circumstances, maintain/slow the deterioration of) their health. By way of example, a patient health plan may provide a patient with one or more of: an exercise plan; a diet/eating plan; a quit smoking plan.

As can be seen, in some cases the patient health plan may be aligned with the patient action items: e.g. if a determined patient action item is to quit smoking, the patient health plan can include a quit smoking plan.

In many instances, helping a patient understand a particular disease or condition they have/are susceptible to increases the patient's willingness/motivation to take action and address that disease/condition. Education plans, therefore, include one or more content items/links to content items hosted by a content provider system (such as CPS 190) that provide information on diseases or conditions that analysis of the patient health data at 314 indicates are or could be relevant to the patient. For example, if analysis of a patient's health data indicates that the patient has or is susceptible to diabetes, the education plan generated may include content items (or links thereto) that explain diabetes, how to manage it, and/or the consequences of failing to manage it. Where content is provided to a patient via a link, the link is displayed to the patient on their patient system 130 (e.g. via the patient client 132). Activation of the link causes the patient system 130 to access the CPS 190 defined in the link to retrieve the relevant content, which the patient system 130 then displays.

Engagement action outputs are generated when analysis of the patient health data results in a determination that further interaction with a healthcare professional is required or warranted. For example, analysis may show that patient pathology information is required or would assist in performing a better/more comprehensive analysis of the patient's health, in which case an appointment with a pathology collection professional will be necessary.

Alternatively, the analysis outcomes may be such that the patient will be recommended to attend a consultation with a doctor (e.g. a GP)—either an in person consultation or a remote consultation by telephone or video call. Determination that a patient should attend a healthcare professional consultation may be based on one or more of the health scores calculated for the patient. For example, in certain implementations if the patient's overall health score (or a specific health area health score) indicates the patient is at moderate or high risk this triggers a doctor consultation engagement action output.

The generation of an engagement action output may also be triggered by direct healthcare professional interaction (via the healthcare professional's HP client 152). For example where a healthcare professional is consulting with a patient (either in person or by telephone/video call), the professional may determine that the patient should book a further consultation, either with the same healthcare professional (e.g. a follow up consultation) or another healthcare professional (e.g. a specialist, a pathology collection laboratory, or other HP). In this case the healthcare professional can enter this information via an appropriate user interface (generated by the HP client 152), the information being communicated to the HMS server 112 to trigger the generation of an engagement action output (and subsequent processing thereof).

As discussed further below, were an engagement action output is generated the HMS server 112 performs further operations in respect thereof to assist the patient engaging with the required/suggested healthcare professional.

Product order action outputs are generated when analysis of the patient health data results in a determination that the patient requires or could benefit from a healthcare product. A healthcare product may be any relevant product, for example a medicine/pharmaceutical (prescription or otherwise) or other product sold for healthcare purposes (e.g. the types of products generally found in chemists/pharmacists/drug stores).

The generation of a product order action output may also be triggered by direct healthcare professional interaction (via the healthcare professional's HP client 152). For example where a healthcare professional is consulting with a patient (either in person or by telephone/video call), the professional may determine that the patient should be prescribed a particular healthcare product (or confirm a prescription that has automatically been suggested by the HMS 110). In this case the healthcare professional can enter this information via an appropriate user interface (generated by the HP client 152), the information being communicated to the HMS server 112 to trigger the generation of a product order action output (and subsequent processing thereof).

As discussed further below, were a product order action output is generated the HMS server 112 performs further operations in respect thereof to assist the patient in obtaining the required/suggested product.

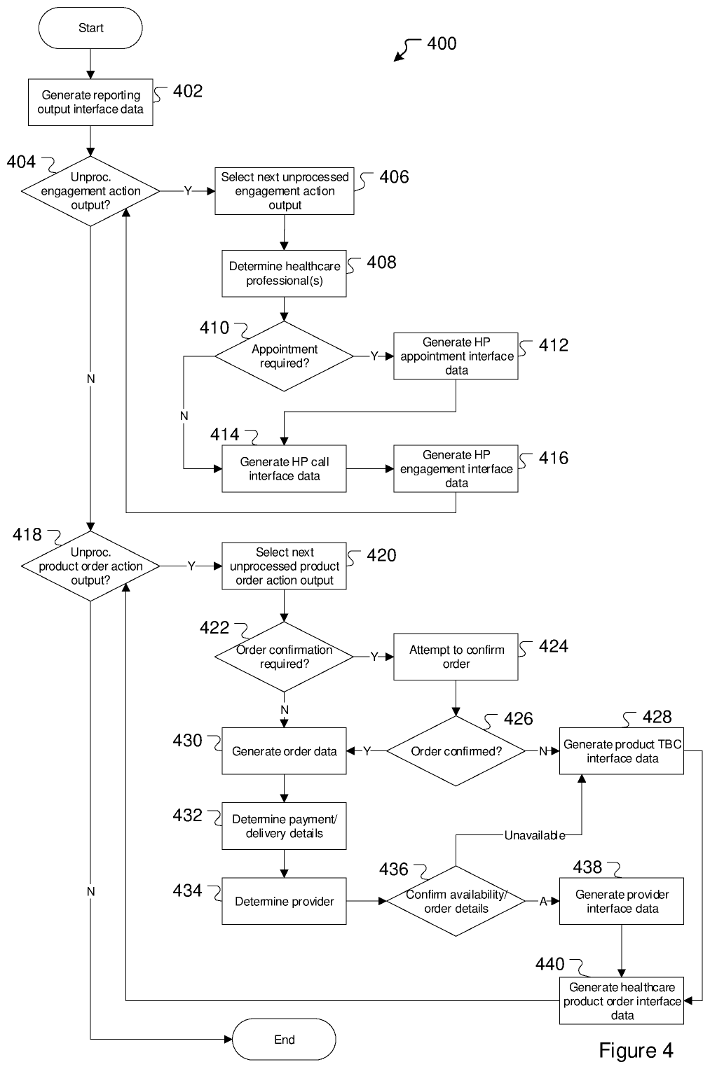

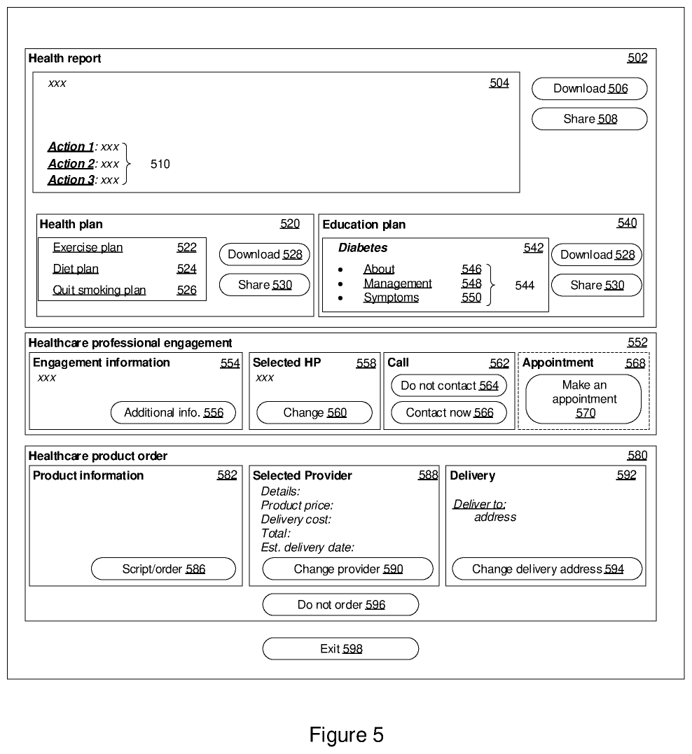

At 316, following analysis of the patient health data and generation of the outputs at 314, the HMS server 112 generates health report interface data and, at 318, communicates this to the patient system 130 (or, more particularly, the patient client 132 running thereon). The health report interface data includes data in respect of reporting and action outputs that have been generated for the patient. The health report interface data is used by the patient client 132 to generate and display a health report interface which provides the patient with the results of their health analysis and, where applicable, assists the patient in furthering their healthcare. Generation of the health report interface data will be described further with reference to FIG. 4, and an example health report interface will be described further with reference to FIG. 5.

In certain cases, in addition to communicating the reporting interface data to the patient client 132, the HMS server 112 communicates some or all of the results of the patient's health analysis to the patient's healthcare professional (i.e. to a HP client 152 of the healthcare professional).

At 320, the HMS server 112 performs further processing with respect to any action outputs provided in the health report interface. This processing is described below with respect to the various controls provided in the example patient interface of FIG. 5. Process 300 then ends.

Process 300 is depicted/described as a single, linear process. In certain embodiments, however, this is not be the case. For example, the health assessment application 116 can be configured to determine the desired patient health data—and, hence, the patient queries—in an adaptive manner. That is, latter queries determined for and presented to a given patient depend on the responses obtained from earlier queries. For example, an early query presented to a patient may ask whether a patient smokes. If the response to that query is affirmative, the health assessment application 116 determines a number of further (latter) questions to ask relevant to smoking and causes these to be presented to the patient (e.g. to establish how long the patient has smoked and how heavily the patient smokes). If, however, the response is that the patient does not smoke these queries are omitted.

Furthermore, in many cases process 300 (or parts thereof) are repeated periodically for a given patient. For example, a patient my access the system on a regular basis (e.g. once every 6 months, once every year, one every 2 years) to update their health information and receive an updated health report. In this case the HMS 110 takes into account previous query responses provided by/on behalf of the patient and any other information (e.g. actions taken on behalf of the patient) when determining which (if any) further queries should be presented to the patient.

As a further example, the health assessment application 116 can be configured to automatically reanalyse a patient's data at certain defined milestones and/or if the underlying health assessment data (and, therefore, patient health data determined to be relevant) changes. For example, if a patient's date of birth shows the patient has just turned 40 and analysis of the patient's health data has not been performed within a determined time period (e.g. within the last 6 months), the HMS 110 may be configured to automatically re-analyse the patient's data taking into account their increased age. Such reanalysis may be done without any further queries provided to the patient, or may involve contacting the patient/patient's healthcare professional (e.g. via the relevant client application 132/152, email, or other means) to acquire further patient health data.

As a further example, new medical research may come to light indicating that x (x being an item of information re a patient's health history, demographic, behavioural habits etc.) is a relevant factor for determining a certain condition/illness. In this case the health assessment data maintained by the HMS 110 maybe updated and (where x has not previously been considered relevant) one or more new queries generated to obtain information in respect of x. In this case, when the HMS 110 is so updated patients can be automatically contacted (e.g. via email or their client applications 132) to provide them with the newly generated queries and obtain information in response thereto.

At 316 and 318 of process 300 described above, the HMS server 112 generates health report interface data and communicates this to the HMS client 132 to enable generation of a health report interface.

This section describes a process for generating of the health report interface data 400 in accordance with an embodiment (with reference to FIG. 4). Following this, an example health report interface 500 will be described with reference to FIG. 5.

At 402, the HMS server 112 generates reporting output interface data. The reporting output interface data includes information based on the reporting outputs generated by the health assessment application 116 (e.g. patient health report, health plan, education plan) at 314. When communicated to the HMS client 132, the reporting output interface data is used by the HMS client 132 to generate a reporting output interface.

Interface 500 of FIG. 5 (described below) provides one example of a reporting output interface 502, from which further details with respect to the reporting output interface data generated by the HMS server 112 at 402 will be apparent.

At 404, the HMS server 112 determines whether any unprocessed health professional engagement action outputs (as generated at 314) exist. If so, processing proceeds to 406. If not, processing proceeds to 418.

At 406, the HMS server 112 selects the next unprocessed health professional engagement action output to process.

At 408, the HMS server 112 determines one or more healthcare professional(s) which will be associated with the selected action output. The HMS server 112 can be configured to make this determination in various ways. For example, in certain embodiments the HMS server 112 is configured to initially determine the type of healthcare provider required for the action output (e.g. a general practitioner) then determine if the patient has defined a preferred healthcare provider for that type of service (as described above). If so, that healthcare professional is selected. If the patient has not defined a preferred provider for the required type of service, the HMS server 112 selects a healthcare professional (or several professionals, in order to provide the patient with options) from the available healthcare professionals (i.e. those having accounts with the HMS 110). In this case the professional(s) may be selected for example, based on their proximity to the patient's work or home address, on whether the patient has previously used one of the professionals, on a rating/feedback score for the professional(s) and/or any other appropriate criteria.

At 410, the HMS server 112 determines whether the current engagement action output requires an appointment (or, conversely, if a telephone or video call will suffice for the matter in question). Whether an appointment is required is indicated in the engagement action output generated by the health assessment application 116, for example by a flag or other variable.

If, at 410, the HMS server 112 determines that an appointment is not required, processing continues to 414. Conversely, if the HMS server 112 determines that an appointment is required, processing continues to 412.

At 412, the HMS server 112 generates HP appointment interface data (descried further below with respect to FIG. 5). Generally speaking HP appointment interface data provides controls that facilitate making an appointment with a healthcare professional (or generates a flag which indicates to the HMS client 132 that such controls should be generated). Processing then continues to 414.

At 414, the HMS server 112 generates HP call interface data (descried further below with respect to FIG. 5). Generally speaking, the HP call interface data provides controls by which the patient can opt-out of or initiate an immediate call from a healthcare professional (or generates a flag which indicates to the HMS client 132 that such controls should be generated).

At 416, the HMS server 112 generates a set of HP engagement interface data. The set of HP engagement interface data includes any general information in respect of the HP engagement (e.g. the reason the engagement has been created), any associated information (e.g. a pathology form where the HP engagement is in respect of pathology collection, a referral where the HP engagement is for a specific issue), data in respect of the selected HP(s) (as determined at 408), HP call interface data (as generated at 414), and HP appointment interface data (generated at 412, if any). When communicated to the HMS client 132, the HP engagement interface data is used by the HMS client 132 to generate a HP engagement interface.

Interface 500 of FIG. 5 provides one example of a HP engagement interface 552, from which further details with respect to the HP engagement interface data generated by the HMS server 112 at 416 will be apparent.

Following generation of the HP engagement interface data at 416, processing returns to 404 to determine whether any further unprocessed HP engagement action outputs exist.

If, at 404, the HMS server 112 determines that no unprocessed health professional engagement action outputs exist, processing proceeds to 418.

At 418, the HMS server 112 determines whether any unprocessed product order action outputs (as generated at 314) exist. If so, processing proceeds to 420. If not, processing ends.

At 420, the HMS server 112 selects the next unprocessed product order action output to process.

At 422, the HMS server 112 determines whether confirmation of the healthcare product defined in the current action output is required. In certain cases—e.g. certain products in certain regions or where the generation a product order action has been triggered by a healthcare professional—confirmation of a healthcare product order may not be required. In other cases—for example where the products are restricted sale pharmaceuticals which can only be prescribed by approved healthcare practitioners—confirmation will be required. Whether confirmation is required is indicated in the product order action output, for example by a flag or other variable.

If, at 422, the HMS server 112 determines that confirmation is not required, processing continues to 430. Otherwise, processing continues to 424.

At 424, the HMS server 112 attempts to confirm the healthcare product order. The HMS server 112 may be configured to perform this attempt in various ways. For example, the HMS server 112 may provide the health report that has been generated to a healthcare practitioner who is qualified to confirm the order (e.g. via a HP client application 152) and await for confirmation. In some cases confirmation will not be possible without the healthcare professional actually consulting with the patient in question (either in a face-to-face or telephone/video call consultation). In this case, the product(s) to which the product order action output relates are suggested products only, subject to confirmation by the appropriate healthcare professional. Furthermore, in this case an engagement action output will typically have been generated in order to arrange the required consultation with the healthcare professional.

Following 424, processing continues to 426 where the HMS server 112 determines whether confirmation has been received. In certain embodiments HMS server 112 is configured to wait only a predetermined amount of time for a confirmation. If confirmation of the order is not received, processing continues to 428 where the HMS 112 generates product order to be confirmed interface data. This data, when communicated to a patient HMS client 132, causes generation of a message indicating that confirmation of a healthcare product order is pending. Following generation of the product order to be confirmed interface data processing continues to 440.

If, at 426, confirmation of the order is received, processing continues to 430.

At 430, either no order confirmation is required (per 422) or confirmation is required and has been received (per 426). As described below, 430 may also be effectively reached where a healthcare professional ‘manually’ enters a product order (via their client 152).

At 430, the HMS server 112 generates order data (which may take the form of a script) in respect of the healthcare product to be ordered. Order data can include, for example: a product name; a product identifier; a product quantity; any other relevant product data. Patient parameters (as described above) can impact the order data. For example, where the product is drug/pharmaceuticals and a patient has indicated they prefer to use generic (as opposed to originator/brand-name) products, a generic product is selected for the order.

At 432, the HMS server 112 determines payment and delivery details in respect of the product to be ordered. These details are retrieved from the account/parameter details associated with the patient for whom the order is being prepared.

At 434, the HMS server 112 determines a provider for the product in question. The HMS server 112 can be configured to make this determination in various ways. For example, in certain embodiments the HMS server 112 is configured to fill all orders using a single provider (i.e. product provider system 170)—in which case that supplier is selected. In other embodiments, the HMS server 112 can interface with multiple providers (via their PPSs 170), in which case a particular provider can be selected according to various criteria. E.g. if the patient has defined a preferred product provider (as described above), that provider is selected. If not, a provider may be selected based on price, product availability/estimated delivery date, and/or any other appropriate criteria.

At 436, the HMS server 112 determines whether the selected provider has the product available (and other information such as product price and estimated delivery time to the patient's preferred delivery address). This involves the HMS server 112 communicating with the PPS server 172 of the relevant PPS 170.

If, at 436, the HMS server 112 determines that the selected provider cannot provide the product (e.g. it is out of stock), processing proceeds to 428 to generate product order TBC interface data as described above. Otherwise, processing continues to 438.

In embodiments where multiple providers are available to the HMS server 110, if the provider initially tried at 436 cannot provide the product, other providers may be queried to determine if they can fill the order. In this case, processing proceeds to 428 only if no providers can provide the product.

At 438, the HMS server 112 generates provider interface data. Generally speaking, the provider interface data includes information in respect of the provider and their supply of the product (e.g. the specific product, price, and estimated delivery date). Following 438 processing proceeds to 440.

At 440, following confirmation that a provider can fulfil the order at 436, the HMS server 112 generates product order interface data. The product order interface data includes any general information in respect of the healthcare product (e.g. the reason the product has been determined necessary/desirable for the patient), product TBC interface data (if generated at 428), and provider interface data (if generated at 438). When communicated to the HMS client 132, the product order interface data is used by the HMS client 132 to generate a product order interface.

Interface 500 of FIG. 5 provides one example of a product order interface 580, from which further details with respect to the product order interface data generated by the HMS server 112 at 440 will be apparent.

Following generation of the healthcare product order interface data at 440, processing returns to 418 to determine whether any further unprocessed product order action outputs exist.

If, at 418, the HMS server 112 determines that no unprocessed product order action outputs exist, processing ends. The interface data at this point includes reporting output interface data generated at 402, any (i.e. zero or more) sets of HP engagement interface data generated at 416, and any (i.e. zero or more) sets of product order interface data generated at 440. The interface data is communicated by the HMS server 112 to the client application 132 at 318.

In many cases, confirmation of a product order (where required) will not occur in real time because the healthcare required to confirm the order either needs to see the patient or, even if not, cannot immediately action the confirmation request. In this case product order to be confirmed interface data is generated (so as to not hold up the reporting process). If the healthcare professional does eventually approve the order, separate processing (e.g. operations 430, 432, 434, 436, and 438) can be performed solely for generation of product order interface data which can then be communicated to the patient's client application 132 to cause display of a product order interface as described below.

Furthermore, in some cases a medical product order action output may be generated by a healthcare professional on seeing a patient (rather than as a result of analysis of the patient's health data by the HMS 110). In this cases the healthcare professional can interact with the HMS server 112 (via their client 152) to enter ender a product order action output (or similar action item). When received by the HMS server 112, the output/item created by the healthcare professional is processed (e.g. per operations the same as or similar to 430, 432, 434, 436, and 438) in order to generate product order interface data. The product order interface data generated can then be communicated by the HMS sever 112 to the patient's client application 132 to cause display of a product order interface as described below.

Interface 500 of FIG. 5 provides one example of how the various interface data generated by the HMS server 112 and communicated to a HMS client 132 is displayed by the HMS client 132 on a patient system 130. Generally speaking, interface 500 includes a reporting output interface 502, a HP engagement interface 552, a healthcare product order interface 580, and an exit control 598 (which, when activated by a patient, exits the interface).

Reporting output interface 502 includes a health report display pane 502, a health plan display pane 520, and an education plan display pane 540, each of which will be described in turn.

Health report display pane 502 is used to display patient health report information, in this instance including content 504 (e.g. text, images, or other content) in respect of the patient's health. All patient health report information can be displayed at once, or the information can be presented in an interactive manner—for example with certain summary items initially displayed along with user controls (such as hyperlinks) which, when activated by a user, cause the presentation of more detailed information. In addition, various controls can be provided, for example a download health report control 504 (which, when activated, allows the patient to download their health report to their patient system 130) and a share health report control 506 (which, when activated, allows the patient to share their health report with selected recipients, for example by email, SMS, or any other appropriate means—recognising the potentially sensitive nature of the information that may be in the health report).

Health report display pane 502 of the present example also includes three patient action items 510—i.e. the top n (in this example 3) patient actions that will have beneficial impact on the patient's health.

In example interface 500, patient health plan information is provided in a health plan display pane 520. In this particular example, three health plan links are displayed: an exercise plan link 522, a diet plan link 524, and a quit smoking plan link 526. Each of these links causes, when activated by a patient, results in further information on the plan in question to be displayed (the further information either received from the HMS server 112 at 318 or retrieved from the HMS 112 at the time the link is activated). In alternative embodiments, health plan information may be set out in full in the health plan display pane 520. Health plan display pane 520 also includes various controls, in this case a download health plan control 528 (which, when activated, allows the patient to download their health plan(s) to their patient system 130) and a share health plan control 530 (which, when activated, allows the patient to share their health plan(s) with selected recipients).

In example interface 500, patient education plan information is provided in an education plan display pane 540. In this particular example, a single disease/condition pane 542 is illustrated (diabetes) which includes three education links to three separate aspects of the disease/condition: an about link 546 (linking to information about the disease/condition); a management link 548 (linking to information about managing the disease/condition); and a symptoms link 550 (linking to information about symptoms of the disease/condition and how those symptoms may change depending on how the disease/condition is managed/mismanaged). Additional, fewer, and/or alterative education can be provided, linking to different (or differently presented) information concerning the disease/condition, and additional disease/condition panes may be displayed. Education links 546, 548, and 550 can link to any appropriate content source, for example the HMS server 112 where education content is maintained by the HMS 110 or a content provider system 190 (more specifically a CPS server 192 thereof) if the education content is maintained by a third party content provider.

HP engagement interface 552 includes an information pane 554 which provides general information in respect of the suggested engagement. This can include, for example, the reason for the engagement and any other relevant information associated therewith. Information pane 554 further includes an additional information control 556 which, if activated by a patient, allows for additional information in respect of the engagement to be accessed (e.g. displayed, downloaded, or otherwise accessed). For example, if the healthcare professional engagement is in respect of a pathology appointment, the additional information can include a pathology form that the patient can download/print and provide to a pathology provider. If the healthcare professional engagement is in respect of a general practitioner (GP) appointment, the additional information can include a referral with information that may be relevant for the appointment.

HP engagement interface 552 also includes a selected healthcare professional pane 558 which includes information on the healthcare professional selected by the HMS server 112 at 408. In this case a single healthcare professional has been selected by the HMS server 112.

The selected healthcare professional pane 558 also includes a change healthcare professional control 560. If a patient wishes to select an alternative healthcare professional for the engagement in question, he or she can activate control 560. This causes a list of alternative healthcare professionals to be displayed (the alternative healthcare professionals either received from the HMS server 112 initially or retrieved therefrom on activation of control 560). The patient can then select an alternative healthcare professional for the engagement (the selection being communicated back to the HMS server 112).

In other cases, the selected healthcare professional pane 558 can display a list of two or more healthcare professionals selected by the HMS server 112 at 408. In this case the user can select one of the displayed healthcare professionals (the selection being communicated back to the HMS server 112).

If the selected HP is changed after the HMS server 112 queues a patient call (as discussed below), the HMS server 112 cancels the patient call queued with the previous HP (also discussed below) and queues a patient call with the newly selected HP.

HP engagement interface 552 also includes a call interface 562. Call interface 562 includes a do not contact control 564 and a contact now control 566.

If the HMS server 112 determines that neither the do not contact control 564 nor the contact now control 566 is activated, the HMS server 112 causes a patient contact reminder to be generated for the selected healthcare professional.

The HMS server 112 is configured to determine that neither the do not contact control 564 nor the contact now control 566 has been activated if the session with the patient client 132 is terminated without receiving an indication from the patient client 132 that either control was activated. In certain implementations, the HMS server 112 is also configured to determine that neither the do not contact control 564 nor the contact now control 566 has been activated if it does not receive an indication from the patient client 132 that either control was activated within a predetermined time period (for example 15 minutes or any other appropriate time period).

The HMS server 112 can be configured to cause a patient contact reminder to be generated for the selected healthcare professional in various ways. For example, in some embodiments the HMS server 112 generates and sends a queue call (or queue contact) message (including, for example, the name and number of the patient to be called and any associated information—for example whether the call is simply to arrange an appointment or to discuss details regarding the patient) to the HP client application 152. The HP client application 152 receives the queue call message and causes a call for the patient to be queued in a call queue of the healthcare professional. In alternative embodiments, the HMS server 112 is configured to send a calendar invitation (e.g. by email, instant message, or other means) to the healthcare professional including call details. When the calendar invitation is received by the healthcare professional it can be accepted and the call is added to the healthcare professional's calendar. By way of still further example, the HMS server 112 may be configured to directly access a call queue/diary/calendar of the healthcare professional (via appropriate API) and add a call item directly thereto.

The HMS server 112 can be configured to determine that neither the do not contact control 564 nor the contact now control 566 was activated on either a timeout (e.g. x seconds/minutes after display of the interface has occurred without either control being activated) or if the session with the client application 132 is closed (e.g. by activation of exit control 598 or termination of the HMS client application 132) without either control being activated.

If the do not contact control 564 is activated, the HMS client 132 generates a do not contact message and communicates this to the HMS server 112. On receipt of a do not contact message from the HMS client 132, the HMS server 112 records the patient's active election not to receive a call and ensures that no call item is placed in the healthcare professionals call queue. If a do not contact message is received after the HMS server 112 has already generated/sent a queue call message, the HMS server 112 takes further action on receiving the do not contact message to attempt to remove the contact reminder from the healthcare professional's call queue. The HMS server 112 can be configured to do this in various ways. For example, in some embodiments the HMS server 112 does so by generating and sending a cancel queued call (or cancel queued contact) message (including, for example, the name and number of the patient to be called and/or other information allowing the queued call to be identified) to the HP client application 152. The HP client application 152 receives the cancel queued call message and removes any queued call for that patient.

In certain embodiments, if the do not contact control 564 is activated the HMS server 112 triggers a patient reminder process which operates to send the patient one or more reminders that a call/consultation with a healthcare professional is recommended.

By way of example, the patient reminder process may involve automatically generating and sending the patient a first reminder a first predetermined period after the client activates the do not contact control (e.g. at one week). If, at a second predetermined period (e.g. 2 weeks after activation of the do not contact control) no data indicating that the patient has been in contact with a healthcare professional has been received by the HMS server 112, a second reminder is generated and communicated to the patient. If, at a third predetermined period (e.g. 4 weeks after activation of the do not contact control) no data indicating that the patient has made been in contact with a healthcare professional has been received by the HMS server 112, a third (and in this particular example final) reminder is generated and communicated to the patient. A given reminder may be communicated to the patient by various means, for example email, SMS, and/or via the patient client application 132. To reduce the likelihood of the patient missing the reminders, reminders may be sent through multiple channels—e.g. the first reminder by email, the second by SMS, and the third by email.

If the contact now control 566 is activated, the HMS client 132 initiates a call with the currently selected healthcare professional (as displayed in pane 558). The call may be a voice call or video call, and is initiated using contact information of the healthcare professional. In this case the HMS client 132 also generates a call placed message and communicates this to the HMS server. On receipt of a call placed message from the HMS client 132, the HMS server 112 records that a call was placed and ensures that no call item is placed in the healthcare professionals call queue (or, if a contact reminder had already been placed in the healthcare professional's call queue, it is removed).

HP engagement interface 552 also includes an appointment interface 568. In certain embodiments, appointment interface 568 is only displayed in the event that an appointment is needed for the engagement in question. In other embodiments an appointment interface 568 is displayed regardless of whether an appointment determined to be necessary by the HMS 110 or not. In this example, appointment interface 568 includes a make appointment control 570. As noted above, in some cases an appointment will be for a face-to-face consultation, while in other cases an appointment may be for a telephone or video call consultation.

If the appointment control 570 is activated, an appointment interface (not shown) is displayed, allowing the patient to view the HPs availability and select an appointment date/time. The HPs appointment schedule can be obtained/interacted with in various ways. For example, if the HPs appointment schedule is publicly available (e.g. via a website or application), the HMS client 132 can be configured to redirect to the healthcare professional's schedule to allow the patient the make an appointment. Alternatively, the HP may provide access to its appointment schedule to the HMS server 112 (via appropriate API). In this case, the HMS client 132 communicates a manual appointment request to the HMS server 112 which, on receipt, accesses the HPs schedule and provides information in respect thereof to the HMS client 132 for display to the patient. The patient can then select a desired appointment time.

If an appointment is made, the HMS server 112 ensures that no call item is placed in the healthcare professionals call queue (or, if a contact reminder had already been placed in the healthcare professional's call queue, it is removed).

As will be appreciated, where analysis of the patient's health data results in further engagement with a healthcare professional being suggested, the default operation is such that a call item will be placed in a healthcare professional's queue for the patient (and, absent some error on the healthcare professional's part, the patient will receive a call). Phrased alternatively, the patient must take positive action to prevent being called by a healthcare professional. This reduces the work required by the patient to further their healthcare.

In alternative embodiments, the default operation may be not to have a call queued for the patient and instead require some explicit action by the patient to make this happen. In this case, rather than displaying a do not contact control 564, the call interface 562 is provided with a queue patient call control. If the queue patient call control is activated the HMS client 132 generates a queue patient call message and communicates this to the HMS server 112 (which then causes a patient call to be queued as discussed above). Even in this case the work/interaction required by the patient to have a healthcare professional call them reduced, as all the patient needs to do is activate a single ‘queue patient call’ control. In such embodiments, a ‘queue patient call’ control can be considered an ‘approve contact’ control—i.e. a control which is activated by a patient to approve contact by a healthcare professional. A ‘contact now’ control such as 566 and/or an appointment control such as 570 as described above are also types of ‘approve contact’ controls in that their activation indicates the patient approves contact with the healthcare professional. In embodiments that require some explicit action by the patient to approve communication with a healthcare professional, if an approve contact control is not activated by the patient, the HMS server 112 may be configured to trigger a patient reminder process as described above—e.g. a process which operates to cause one or more reminders to be sent to the patient to remind the patient that a call/consultation with a healthcare professional is recommended.

Healthcare product order interface 580 includes a product information pane 582 which provides general information in respect of the healthcare product(s) that the order interface 580 relates to. This can include, for example, information on: the name(s) of the product(s) the order interface relates to; the reason the product(s) has/have been identified for the patient; any usage suggestions/requirements associated with the product(s); any other relevant information. The product information pane also includes a script/order control 586 which, if activated by a patient, can be configured to cause a script or order document for the product(s) to be displayed/downloaded and/or shared. This is useful if the patient elects not to proceed with an electronic order for the product but instead wants to take the script/order into a physical store.

Healthcare product order interface 580 also includes a selected provider pane 588 which includes information on the product provider selected by the HMS server 112 at 434. In this example the information includes details of the provider (e.g. the name of the name of the selected product provider, such as Amazon, Chemist Warehouse, or whichever provider has been selected); the price of the product(s); the delivery cost to the currently selected delivery address; the total cost of the order; and the estimated delivery date of the product(s). Additional/less/alternative provider information can be provided.

Selected provider pane 588 also includes a change provider control 590. If a patient wishes to select an alternative provider for the product(s), he or she can activate control 590. This causes a list of one or more alternative providers available to the HMS 110 to be displayed (the alternative providers either received from the HMS server 112 initially or retrieved therefrom on activation of control 590). The patient can then select an alternative provider from the list, causing a change provider message to be generated by the client 132 and communicated back to the HMS server 112. On receipt of a change provider message, the HMS server 112 interfaces with the PPS server 172 of the newly selected provider to check that the supplier can supply the product(s) in question (e.g. per 436 described above). If so, the HMS server 112 generates a provider update message and communicates this to the HMS client 132. The provider update message includes updated provider data which the HMS client 132 causes to be displayed in the selected provider pane 588 (e.g. the newly selected provider details, any change in product price, any change in delivery cost and/or estimated delivery date).

Healthcare product order interface 580 also includes a delivery address pane 592 which provides information on the currently selected delivery address (as determined at 432). The delivery address pane 592 also includes a change delivery address control 594. If a patient wishes to select or enter an alternative delivery address, he or she can activate control 594. This causes a change delivery address interface (not shown) to be launched. The change delivery address interface can include a data entry field (or series of fields) allowing the user to specify the desired delivery address. If the patient has provided the HMS 110 with multiple addresses (e.g. a work address, home address, etc.), the change delivery address interface can also display these (e.g. by the HMS client 132 requesting stored addresses for the patient from the HMS server 112 and receiving address information in response), allowing the patient to select an alternative address from a list. Selection of an alternative delivery address causes the HMS client 132 to generate a change delivery address message and communicate this message back to the HMS server 112. On receipt of a change delivery address message, the HMS server 112 records the changed delivery address (and, in certain implementations, adds the new delivery address to the patient's account). The HMS server 112 also interfaces with the PPS server 172 of the currently selected provider to determine whether the new delivery address causes any change to the delivery cost or estimated delivery date. On receiving a response from the PPS server 172, the HS server 112 generates a new delivery address message and communicates this to the HMS client 132. The new delivery address message confirms the delivery address has been updated (so the HMS client 132 can cause the updated delivery address details to be changed in the delivery address pane 192). The new delivery address message also includes updated provider data which the HMS client 132 causes to be displayed in the selected provider pane 588 (e.g. any change in delivery cost or estimated delivery date).

Healthcare product order interface 580 also includes a do not order control 596. If the do not order control 596 is not activated, the HMS server 112 causes an order to be placed with the selected provider.

The HMS server 112 is configured to determine that the do not order control 596 has not been activated if the session with the patient client 132 is terminated without receiving an indication from the patient client 132 that the control was activated. In certain implementations, the HMS server 112 is also configured to determine that the do not order control 596 has not been activated if it does not receive an indication from the patient client 132 that the control was activated within a predetermined time period (for example 15 minutes or any other appropriate time period).

The HMS server can be configured to cause an order to be placed with the selected provider in various ways. For example, in some embodiments the HMS server 112 does so by causing the PPS application 114 to place the order with the PPS server 172 of the selected provider. The order placed includes information on the selected product(s), the selected delivery address, and payment details (e.g. credit card or other payment account details retrieved from the patient's account). If the order is successfully placed, the HMS 110 generates an order placed message and communicates this to the patient—e.g. to be displayed by the HMS client 132, or via email or other communication means. If the order is not successfully placed (e.g. because the supplier sold out of the product(s) prior to trying to place the order with the PPS system 172), an order not placed is communicated to the patient. The HMS server 112 can be configured to determine that the do not order control 596 was not activated on either a timeout (e.g. x seconds/minutes after display of the interface has occurred without the control being activated) or if the session with the client application 132 is closed (e.g. by activation of exit control 598 or termination of the HMS client application 132) without the control being activated.

If the do not order control 596 is activated, the HMS client 132 generates a do not order message and communicates this to the HMS server 112. On receipt of a do not order message from the HMS client 132, the HMS server 112 records the patient's active election that the order should not be placed and does not place the order. If a do not order message is received after the HMS server 112 has already placed an order, the HMS server 112 takes further action on receiving the do not order message to attempt to remove/cancel the placed order from the provider system. The HMS server 112 can be configured to do this in various ways. For example, in some embodiments the HMS server 112 does so by generating and sending a cancel order message (including sufficient information to allow the original order to be identified) to the PPS application 114. The PPS application 114 receives the cancel order message and, if possible, cancels the order.

As will be appreciated, where a healthcare product is required or recommended for the patient, the default operation is such that an order for that product will be placed. Phrased alternatively, the patient must take positive action to prevent an order being placed. This reduces the work required by the patient to further their healthcare.

In alternative embodiments, instead of placing an order absent explicit patient action, the default operation may be not to place an order. In this case, rather than displaying a do not order control 596, the healthcare product order interface 580 includes a confirm order control. If the confirm order control is activated the HMS client 132 generates an order confirmed message and communicates this to the HMS server 112 (which then causes the order to be placed as described above). If the confirm order control is not activated no order is placed. Even in this case the work/interaction required by the patient to order prescribed/suggested healthcare products is reduced, as all the patient needs to do in order to have the order placed is activate a single ‘confirm order’ control.

In some cases an order cannot be placed—for example the product(s) are simply not available from any product provider the HMS 110 can access, or the order is not confirmed at 426. In this case, the product information pane 582 may still be displayed, but instead of displaying a selected provider pane 588 or delivery address pane 592 an appropriate message is displayed—for example “products unavailable” or “awaiting healthcare professional confirmation of order” or other appropriate message.

While the operations described above with reference to FIGS. 3, 4, and 5 have been described as being performed by specific applications, the described operations could be performed by any appropriately programmed/configured application. For example, in certain implementations the HMS 110 can be configured to perform its operations by a single, monolithic application running thereon rather than by separate applications.

The flowcharts of FIGS. 3 and 4 have been provided and described in order to illustrate processing/functional steps. Although these flowcharts define steps in particular orders to explain various features, in some cases the steps may be able to be performed in a different order or in parallel. Furthermore, in some cases two or more operations may be combined into a single operation, a single operation may be divided into multiple separate operations, and/or the function(s) achieved by one or more of the described/illustrated operations may be achieved by one or more alternative operations.

It will be understood that the embodiments disclosed and defined in this specification extend to all alternative combinations of two or more of the individual features mentioned or evident from the text or drawings. All of these different combinations constitute various alternative aspects of the embodiments.

1. A computer implemented method including:
receiving, by a health management system, patient health data in respect of a patient;
analysing, by the health management system, the patient health data to determine that engagement with a particular type of healthcare professional is required;
determining, by the health management system, a specific healthcare professional of the particular type;
causing, by the health management system, a healthcare professional engagement interface to be generated, the healthcare professional engagement interface including information indicating that engagement with the particular type of healthcare professional is required and a do not contact control;
in response to determining that the do not contact control is not activated, causing a patient contact reminder to be generated for the specific healthcare professional, the patient contact reminder being a reminder for the specific healthcare professional to contact the patient.

2. The computer implemented method of claim 1, wherein in response to determining that the do not contact control is activated, the health management system is configured to forego causing a patient contact reminder to be generated.

3. The computer implemented method of claim 1, wherein in response to determining that the do not contact control is activated, the method further includes:
determining, by the health care management system, if a patient contact reminder has already been caused to be generated; and
in response to determining that a patient contact reminder has already been caused to be generated, generating by the health care management system a cancel patient contact message and communicate the cancel patient contact message to the healthcare professional system.

4. The computer implemented method of claim 1, wherein in response to determining that the do not contact control is activated, the method further includes generating, by the health care management system, a patient reminder and communicating the patient reminder to the patient at a predetermined time.

5. (canceled)

6. The computer implemented method of claim 1 wherein the healthcare professional engagement interface further includes a change healthcare professional control, and wherein on detecting activation of the change healthcare professional control the method further includes:
determining, by the health care management system, an alternative healthcare professional suitable for the engagement; and
in response to determining that the do not contact control is not activated, causing a patient contact reminder to be generated for the alternative healthcare professional.

7. The computer implemented method of claim 6, wherein determining an alternative healthcare professional includes receiving, by the health care management system, information in respect of an alternative healthcare professional selected by the patient.

8. The computer implemented method of claim 1 wherein causing a patient contact reminder to be generated includes causing a patient call item to be added to a call queue maintained by or on behalf of the healthcare professional.

9. The computer implemented method of claim 1 wherein causing a patient contact reminder to be generated includes sending, by the health care management system, a calendar invitation to the healthcare professional.

10. The computer implemented method of claim 1, wherein causing a patient contact reminder to be generated includes adding, by the health care management system, a calendar event directly into a calendar of the healthcare professional.

11. A computer implemented method including:
determining, by a health management system, that a patient requires a healthcare product;
determining, by the health management system, a specific provider for the healthcare product;
causing, by the health management system, a healthcare product order interface to be generated, the healthcare product order interface including information regarding the healthcare product and a do not order control;
in response to determining that the do not order control is not activated, generating a product order and communicating the product order to a provider system of the specific provider.

12. The computer implemented method of claim 11, where determining that the patient requires a healthcare product includes:
receiving, by the health management system, patient health data in respect of a patient; and
analysing, by the health management system, the patient health data to determine that the patient requires the healthcare product.

13. The computer implemented method of claim 11, where determining that the patient requires a healthcare product includes receiving, by the health management system, a communication from a healthcare professional system indicating that the patient requires the healthcare product.

14. The computer implemented method of claim 11, wherein in response to determining that the do not order control is activated, the health management system is configured to forego generation of a product order and communication of the product order to the provider system.

15. The computer implemented method of claim 11 wherein in response to determining that the do not order control is activated, the method further includes:
determining if a product order has already been generated and communicated to the provider system; and
in response to determining that a product has already been generated and communicated to the provider system, generating a cancel order message and communicating the cancel order message to the provider system.

16. The computer implemented method of claim 11 wherein prior to generating and communicating the product order the method further includes:
determining if confirmation is required prior to ordering the healthcare product for the patient; and
in response to determining that confirmation is required, foregoing generation of the product order unless confirmation is obtained.

17. The computer implemented method of claim 16, wherein the healthcare product order interface further includes a change provider control, and wherein on detecting activation of the change provider control the method further includes:
determining an alternative provider; and
in response to determining that the do not order control is not activated, generating a product order and communicating the product order to a provider system of the alternative provider.

18. (canceled)

19. A computer implemented method including:
receiving, by a health management system, patient health data in respect of a patient;
analysing, by the health management system, the patient health data to determine that engagement with a particular type of healthcare professional is required;
determining, by the health management system, a specific healthcare professional of the particular type;
causing, by the health management system, a healthcare professional engagement interface to be generated, the healthcare professional engagement interface including information indicating that engagement with the particular type of healthcare professional is required and one or approve contact controls;
in response to determining that none of the one or more approve contact controls is activated, generating, by the health care management system, a first patient reminder and communicating the first patient reminder to the patient at a first predetermined time.

20. The computer implemented method of claim 19, wherein the method further comprises:
determining, at a second predetermined time, that the patient has not contacted a healthcare professional and, in response, generating a second patient reminder and communicating the second patient reminder to the patient.

21. The computer implemented method of claim 20, wherein the first and second patient reminders are sent through multiple communication channels.

22. The computer implemented method of claim 19, wherein:
The one or more approve contact controls include one or more of: a queue patient call control, a contact now control; and a make appointment control.

23. A system including:
one or more processors;
one or more non-transitory computer-readable storage media storing sequences of instructions which, when executed by the one or more processors, cause the one or more processors to:
receiving, by a health management system, patient health data in respect of a patient;
analyse, by the health management system, the patient health data to determine that engagement with a particular type of healthcare professional is required; determine, by the health management system, a specific healthcare professional of the particular type;
cause, by the health management system, a healthcare professional engagement interface to be generated, the healthcare professional engagement interface including information indicating that engagement with the particular type of healthcare professional is required and a do not contact control; and
in response to determining that the do not contact control is not activated, cause a patient contact reminder to be generated for the specific healthcare professional, the patient contact reminder being a reminder for the specific healthcare professional to contact the patient.

24. (canceled)
